(19) United States
(12) Patent Application Publication
(10) Pub. No.: US 2010/0196327 A1
(43) Pub. Date: Aug. 5, 2010

(54) METHODS FOR DIAGNOSING BIOLOGICAL SAMPLES CONTAINING STEM CELLS

(75) Inventor: Roger Deutsch, Highland Beach, FL (US)

Correspondence Address: Gregory A. Nelson;Novak Druce & Quigg LLP, 525 Okeechobee Blvd, Suite 1500, West Palm Beach, FL, 33401, US

(73) Assignee: Cell Science Systems, Deerfield Beach, FL (US)

(21) Appl. No.: 12/595,777

(22) PCT Filed: Apr. 11, 2008
(86) PCT No.: PCT/US08/60069

§ 371 Date: Mar. 26, 2010

Related U.S. Application Data
(60) Provisional application No. 60/923,091, filed on Apr. 11, 2007.

(51) Int. Cl. A61K 35/12; G01N 33/567; C12Q 1/02; C12N 5/00; C12N 5/02; C12M 1/34

(52) U.S. Cl. 424/93.7; 435/7.21; 435/29; 435/325; 435/375; 435/287.1

(57) ABSTRACT

The present invention relates to a method for diagnosing the compatibility of a biological sample containing stem cells from a donor with the immune system of a recipient. Furthermore, the present invention relates to a method for determining the quality of a stem cell preparation based on the inventive method, as well as methods of diagnosing an immune disorder affecting stem cell recognition. The present invention further relates to a method for producing an improved stem cell preparation, and an apparatus that is equipped for performing the method according to the invention. The invention can be used in the field of stem cell-based transplantation and respective diseases. The invention also includes a method for testing the efficacy of donor immune cells as treatments for disease such as cancer in a host patient. A method for testing the immune response of a patient to recall antigens is also disclosed.

### BACKGROUND OF THE INVENTION

The present invention relates to a method for diagnosing the compatibility of a biological sample containing stem cells from a donor with the immune system of a recipient. Furthermore, the present invention relates to a method for determining the quality of a stem cell preparation, as well as methods of diagnosing an immune disorder affecting stem cell recognition. The present invention further relates to a method for producing an improved stem cell preparation, and an apparatus that is equipped for performing the method according to the invention. The invention can be used in the field of stem cell-based transplantation and respective diseases, and for testing mature somatic cells or the cells of blood relative(s) of donor.

Stem cells are unspecialized cells that have two important characteristics that distinguish them from other cells in the body. First, they can replenish their numbers for long periods through cell division. Second, after receiving certain chemical signals, they can differentiate, or transform into specialized cells with specific functions, such as a heart cell or nerve cell.

Stem cells can be classified by the extent to which they can differentiate into different cell types: a) Totipotent stem cells (e.g. Zygote (fertilized egg), blastomere) can differentiate into any cell type in the body plus the placenta, which nourishes the embryo. A fertilized egg is a type of totipotent stem cell. Cells produced in the first few divisions of the fertilized egg are also totipotent. b) Pluripotent stem cells (e.g. cultured human ES cells) are descendants of the totipotent stem cells of the embryo. These cells, which develop about four days after fertilization, can differentiate into any cell type, except for totipotent stem cells and the cells of the placenta. c) Multipotent stem cells are descendents of pluripotent stem cells and antecedents of specialized cells in particular tissues. For example, hematopoietic stem cells, which are found primarily in the bone marrow, give rise to all of the cells found in the blood, including red blood cells, white blood cells, and platelets. Another example is neural stem cells, which can differentiate into nerve cells and neural support cells called glia. d) Progenitor cells (or unipotent stem cells) can produce only one cell type. For example, erythroid progenitor cells differentiate into only red blood cells.

Research is now being conducted on both adult and embryonic stem (ES) cells to determine the characteristics, and the potential of both to cure disease. Furthermore, other markers of activation of host immune cells are also under investigation.

U.S. Pat. No. 5,672,346 discloses human pluripotent hematopoietic stem cell (PHSC) enriched compositions and methods for obtaining and using the compositions.

Currently, the potential of stem cells to regenerate organs and tissues is under extensive research in order to develop respective treatments. Medical researchers believe that stem cell therapy has the potential to radically change the treatment of human disease. A number of adult stem cell therapies already exist, particularly bone marrow transplants that are used to treat leukaemia. In the future, medical researchers anticipate being able to use technologies derived from stem cell research to treat a wider variety of diseases including cancer, Parkinson's disease, spinal cord injuries, diabetes, and muscle damage, amongst a number of other impairments and conditions.

Organ regeneration has long been believed to be through organ-specific and tissue-specific stem cells. Hematopoietic stem cells were believed to replenish blood cells, stem cells of the gut to replace cells of the gut and so on. Recently, using cell lineage tracking, stem cells from one organ have been discovered that divide to form cells of another organ. Hematopoietic stem cells can give rise to liver, brain and kidney cells. This plasticity of adult stem cells has been observed not only under experimental conditions, but also in people who have received bone marrow transplants. Tissue regeneration is achieved by two mechanisms: (1) Circulating stem cells divide and differentiate under appropriate signalling by cytokines and growth factors, e.g. blood cells; and (2) Differentiated cells which are capable of division can also self-repair, e.g. hepatocytes, endothelial cells, smooth muscle cells, keratinocytes and fibroblasts. These fully differentiated cells are limited to local repair. For more extensive repair, stem cells are maintained in the quiescent state, and can then be activated and mobilized to the required site. For wound healing in the skin, epidermal stem cells and bonemarrow progenitor cells both contribute.

The existence of hematopoietic stem cells was discovered in the 1960s, followed by the discovery of stromal cells (also called mesenchymal cells). Only in the 1990s did scientists confirm the reports of neural stem cells in mammalian brains. Since then stem cells have been found in the epidermis, liver and several other tissues. Adult stem cells offer hope for cell therapy to treat diseases in the future because ethical issues do not impede their use. In addition, if the patient's own cells are used, immunological compatibility is not an issue. However, ES cells have been found to be superior for both differentiation potential and ability to divide in culture. Cord blood, from the umbilical cord, was believed to be an alternate source of hematopoietic stem cells; however, it is impossible to obtain sufficient numbers of stem cells from most cord blood collections to engraft an adult of average weight. Development continues on techniques to increase the number of these cells ex vivo. Cord blood contains both hematopoietic and non-hematopoietic stem cells. An advantage of cord blood is that the stem cells do not show a significant sense of “self” or a generation of a large number of antibodies, so the chances of life-threatening graft-versus-host disease (GVHD) is greatly reduced.

Perhaps the best-known stem cell therapy to date is the bone marrow transplant, which is used to treat leukemia and other types of cancer, as well as various blood disorders. Bone marrow transplantation (BMT) is being increasingly used in humans. In genetically identical twins (syngeneic) there are no immunological barriers to BMT, but in other circumstances genetic disparities result in immune-related complications, including graft rejection and GVHD (Gale and Reisner, Lancet, 1986 Jun. 28; (8496):1468-70).

While most blood stem cells reside in the bone marrow, a small number are present in the bloodstream. These multipotent peripheral blood stem cells, or PBSCs, as well as Natural Killer (NK cells) and cytotoxic lymphocytes (CTLs), can be used just like bone marrow stem cells to treat leukemia, other cancers and various blood disorders. Since they can be obtained from drawn blood, PBSCs, NKs and CTLs are easier to collect than bone marrow stem cells, which must be extracted from within bones. This makes PBSCs, NKs and CTLs a less invasive treatment option than bone marrow stem cells. PBSCs are sparse in the bloodstream, however, so collecting enough to perform a transplant can pose a challenge.

More recently, advances made in the area of autologous (in an autologous transplant, the patient's own stem cells are used) BMT have shown that, in cancer patients receiving such transplants, treatment with granulocyte colony-stimulating factor (G-CSF) or other cytokines, such as granulocyte macrophage colony-stimulating factor (GM-CSF) or interleukin-3 (IL-3), leads not only to elevated levels of neutrophils in the peripheral blood, but also to mobilization of pluripotential stem cells from the marrow to the blood. Thus, following induction with G-CSF, it became possible to collect by leukapheresis large numbers of stem cells (Caspar et al., 1993).

U.S. Pat. No. 5,806,529 describes a method for bone marrow transplantation from an HLA-nonmatched donor to a patient which comprises conditioning the patient under a suitable regimen followed by transplant of a very large dose of stem cells which is at least about 3-fold greater than the conventional doses used in T cell-depleted bone marrow transplantation.

The patient is conditioned under lethal or supralethal conditions for the treatment of malignant or non-malignant diseases, or under sublethal conditions for the treatment of non-malignant diseases. The transplant may consist of T cell-depleted bone marrow stem cells and T cell-depleted stem cell-enriched peripheral blood cells from the HLA-nonmatched donor. preferably a relative of the patient, which donor was previously treated with a drug, e.g. a cytokine such as granulocyte colony-stimulating factor (G-CSF).

As can be taken from the above, there are still several obstacles to be overcome in order to develop stem cell therapy into an effective and less dangerous (e.g. without the risk of life-threatening graft-versus-host disease (GVHD)) therapy. In addition, the amount of material available for stem cell therapy in most cases is limited and has to fulfil strict safety and quality requirements. There is therefore a need in the art, to provide new quick and effective methods and devices in order to overcome some of the limitations as presently faced in stem cell therapy.

### SUMMARY OF THE INVENTION

A method for diagnosing the compatibility of a biological sample containing stem cells from a donor with the immune system of a recipient, includes the steps of:
a) providing a biological sample containing stem cells from a putative donor,
b) providing a biological sample containing immune cells from a putative acceptor of the stem cells,
c) combining the samples from a) and b) under conditions that are suitable to allow for a reaction of the stem cells and the immune cells,
d) determining the number of immune cells that have reacted to the stem cells in the putative acceptor sample, and
e) determining the compatibility of the stem cells of the donor for the acceptor from step d).

A method for determining the quality of a stem cell preparation includes the steps of performing the above compatibility diagnostic method, and then determining the quality of a stem cell preparation, at least in part, from the number of immune cells that have reacted to the stem cells in the putative acceptor sample. A method of diagnosing an immune disorder affecting stem cell recognition includes performing the compatibility diagnostic method described above, and then diagnosing the immune disorder, at least in part, from the number of immune cells that have reacted to the stem cells in the putative acceptor sample.

A method for producing an improved stem cell preparation includes performing the compatibility diagnostic method described above, and then selecting a non-reactive or low-reactive sample based, at least in part, on the number of immune cells that have reacted to the stem cells in the putative acceptor sample, and formulating the selected sample into a stem cell preparation. The invention includes a stem cell preparation produced according to this method, and a method for treating a disease, which includes administering to a patient in need thereof an effective amount of the stem cell preparation.

A method for measuring cell reactivity between allogeneic donor and host cells includes the steps of mixing the allogeneic cells, incubating the mixture, and adding to the mixture a cytotoxic agent that is preferential to one of either the host or the donor cells, and then measuring the reactivity of the other of either the host or the donor cells.

A method for measuring the reactivity between allogeneic host and donor cells includes the steps of mixing the host and donor cells and measuring the number of cells, allowing the mixture to incubate for a period of time, and then measuring the cells after the period of time.

A method for determining changes to stem cells as the result of environmental factors includes the steps of exposing the cells to the environmental factors, and measuring the effect of the environmental factors on the cells after the exposure.

A method of treating disease in a host patient includes the steps of providing donor cells selected to have a therapeutic effect on the disease, obtaining diseased cells from the patient, mixing host cells with the therapeutic cells, and measuring the effect of the therapeutic donor cells on the host cells. The invention includes a therapeutic preparation prepared by this method.

A method for measuring the immune response to recall antigens on a patient includes the steps of obtaining a blood sample from said patient, exposing the sample to the recall antigen, and measuring the effect of the exposure on immune cells of the patient in the sample.

### BRIEF DESCRIPTION OF THE DRAWINGS

FIG. 1 is a flow chart showing the steps of the inventive method.

FIG. 2 is a block diagram showing the connection of the various components which make up the present inventive apparatus.

### DETAILED DESCRIPTION OF THE INVENTION

In a first aspect of the present invention, a method is provided for diagnosing the compatibility of a biological sample containing stem cells from a donor with the immune system of a recipient, comprising the steps of a) providing a biological sample containing stem cells from a putative donor, b) providing a biological sample containing immune cells from a putative acceptor of said stem cells, c) combining said samples from a) and b) under conditions that are suitable to allow for a reaction of said stem cells and said immune cells, d) determining the number of immune cells that have reacted to said stem cells in said putative acceptor sample, and e) determining the compatibility of said stem cells of said donor for the acceptor from step d).

If there is an incompatibility of a biological sample containing stem cells from a donor with the immune system of a recipient, this will be indicated according to the present invention by an increase in the number of immune cells that have reacted with said biological sample containing stem cells.

U.S. Pat. No. 5,147,785 (incorporated herewith by reference in its entirety) describes a method for the diagnosis of a malady in a subject by the observing of a degree of reaction between the leukocytes in the subjects blood with a foreign entity having a predetermined relationship with the malady being diagnosed. In said method the number of leukocytes counted in a first unreacted blood sample is compared with a number of unreacted and reacted leukocytes counted in a second blood sample that has been admixed with a substance to be diagnosed, to determine if said leukocytes in said second blood sample reacted with said substance, thereby diagnosing the presence or absence of said malady in said subject. Preferably, said counting of the number of leukocytes of said first blood sample and said mixture is performed by counting a number of leukocytes within each of a plurality of varying size-distribution ranges. The substance can be a foreign entity and/or an antibody.

U.S. Pat. No. 5,147,785 further describes the production of a graph showing said number of leukocytes counted in each of said plurality of size-distribution ranges, as well as the step of lysing a portion of any red blood cells present in said mixture prior to counting said number of unreacted and reacted leukocytes therein and lysing a portion of said reacted leukocytes in said mixture.

According to one further aspect, the invention furthermore comprises determining the number of immune cells that have reacted in said putative acceptor sample before step c) of the method as above. Further preferred is a method according to the present invention, wherein determining the number of immune cells that have reacted in said putative acceptor sample in step d) of the method as above comprises counting a number of unreacted and reacted leukocytes.

According to the present invention, the step of determining the number of immune cells that have reacted in said putative acceptor sample in step d) comprises determining the plurality of varying size-distribution ranges of said cells, for example, and preferably, as described in U.S. Pat. No. 5,147,785. Optionally, a graph showing said number of leukocytes counted in each of said plurality of size-distribution ranges can be prepared as well.

The immune cells can be selected from white blood cells (leukocytes). Nevertheless, also fractions of the sample containing leukocytes can be used, as well as fractions of the types of leukocytes as present in the samples (such as monocytes, macrophages, mast cells, granulocytes, B-cells and/or T-cells).

In a particular embodiment of the method according to the present invention, the stem cells are selected from ES cells, adult stem cells, and/or cells derived from cord blood or amniotic fluid. These particular groups of stem cells currently appear to be most promising approaches for therapeutical approaches (as explained above).

In yet another particular embodiment of the method according to the present invention, the stem cells are of the same or a different HLA-type as the recipient. Allogeneic cells refers to cells taken from different individuals of the same species. Two or more individuals are said to be allogeneic to one another when the genes at one or more loci are not identical. In allogeneic transplants, the donor and recipient must—or should—be “compatible”: their HLA types must be (at least) close. HLA (Human Leukocyte Antigen) type is determined by a blood test (or sometimes DNA testing). HLA are antigens on the surface of a person's cells and are easily found on the leukocytes. These mark the cells as being “of self”. If the antigens do not match, the donated stem cells will attack all cells of the recipient, because they see the recipient as “foreign”. If the antigens match (or nearly match), the donated marrow or cells will peacefully take up residence in the recipient's body and work to supply healthy blood cells. Thus, the HLA-type is essential in order to identify any incompatibility of a biological sample containing stem cells from a donor. Preferably the stem cells are selected from allogeneic, syngeneic or autologous stem cells.

According to a method of the present invention, stem cells are selected from at least one of totipotent, pluripotent, multipotent, oligopotent (i.e. can differentiate into a few types of endoderm, ectoderm, and mesoderm cells), quadripotent (i.e. can differentiate into four types of cells), tripotent (i.e. can differentiate into three types of cells), bipotent (i.e. precursor of two cell types), and unipotent (i.e. precursor of one type of cell) stem cells, as defined herein. Thus, further preferred is a method according to the present invention, wherein the stem cells are selected from at least one of cultured ES cells, hematopoietic cells, myeloid precursor cells, mesenchymal progenitor cells, glial-restricted precursor cells, bipotential precursor cells from fetal liver, umbilical cord blood cells, cells from amniotic fluid, peripheral blood stem cells, bone marrow stem cells, and mast cell precursor cells.

The biological sample containing stem cells comprises ex vivo multiplicated/expanded stem cells. Methods for growth, development and keeping stem cells in culture are described in the literature, such as, for example, in Hofmeister et al. (See Hofmeister CC, Zhang J, Knight K L, Le P, Stiff P J. Ex vivo expansion of umbilical cord blood stem cells for transplantation: growing knowledge from the hematopoietic niche., Bone Marrow Transplant. 2007 January; 39(1):11-23; Smadja D M, Comet A, Emmerich J, Aiach M, Gaussem P. Endothelial progenitor cells: Characterization, in vitro expansion, and prospects for autologous cell therapy. Cell Biol Toxicol. 2007 July; 23(4):223-39; Epub 2007 Mar. 16; Zubler R H. Ex vivo expansion of hematopoietic stem cells and gene therapy development. Swiss Med Wkly. 2006 Dec. 23; 136(49-50):795-9; Metallo C M, Mohr J C, Detzel C J, Pablo J J, Wie B J, Palecek S P. Engineering the stem cell microenvironment. Biotechnol Prog. 2007 January-February; 23(1):18-23; Handschel J, Wiesmann H P, Depprich R, Kubler N R, Meyer U. Cell-based bone reconstruction therapies—cell sources. Int J Oral Maxillofac Implants. 2006 November-December; 21(6):890-8; Fang Y, Orner B P. Induction of pluripotency in fibroblasts through the expression of only four nuclear proteins. ACS Chem Biol. 2006 Oct. 24; 1(9):557-8; and Galvin K A, Jones D G. Adult human neural stem cells for autologous cell replacement therapies for neurodegenerative disorders. NeuroRehabilitation. 2006; 21(3):255-65.), and the references as cited therein.

A method according to the present invention includes a biological sample which comprises a stem cell-derived tissue or organ. This aspect of the present invention relates to an organ or tissue that has been transiently grown or harboured in a host other than the final recipient (e.g. a monkey in the case of a human organ or tissue). Nevertheless, preferred is a method according to the present invention, wherein both the donor and acceptor are humans.

In another aspect of the method according to the present invention, said method further comprises the determination of a stem-cell specific protein expression pattern, in particular of the transcription factors OCT4, SOX2, and/or NANOG. Rao and colleagues (Cai J, Weiss M L, Rao M S. In search of “stemness”. Exp Hematol. 2004 July; 32(7):585-98) postulate that all stem cells, regardless of their origin, share common properties. These researchers have reviewed the literature for candidate “stemness” genes. They conclude that there are a set of candidate genes that are present in all stem cells and can serve as universal markers for stem cells. These code for proteins that are involved in self-renewal and differentiation. In addition they predict some differences in gene expression between different populations of stem cells. Furthermore, Boyer et al. (in Boyer L A, Lee T I, Cole M F, Johnstone S E, Levine S S, Zucker J P, Guenther M G, Kumar R M, Murray H L, Jenner R G, Gifford D K, Melton D A, Jaenisch R, Young R A. Core transcriptional regulatory circuitry in human embryonic stem cells. Cell. 2005 Sep. 23; 122(6):947-56.) describe that the transcription factors OCT4, SOX2, and NANOG have essential roles in early development and are required for the propagation of undifferentiated embryonic stem (ES) cells in culture. The term “sternness” designates the common molecular processes underlying the core stem cell properties of self-renewal and the generation of differentiated progeny.

Yet another aspect of the present invention relates to a method for determining the quality of a stem cell preparation, comprising performing a method as described above, and determining the quality of a stem cell preparation, at least in part, from the number of immune cells that have reacted to said stem cells in said putative acceptor sample. Preferred is a method, wherein said quality is selected from the purity and/or viability of said stem cells, the sternness as defined above, and the stability (homogeneity).

Yet another aspect of the present invention then relates to a method of diagnosing an immune disorder affecting stem cell recognition comprising performing a method according to the invention as above, and diagnosing said immune disorder, at least in part, from the number of immune cells that have reacted to said stem cells in said putative acceptor sample. In this context, the lack of a response and/or a disturbed response (i.e. a response that is higher or lower compared to a sample taken from a non-diseased control patient) indicates an immune disorder affecting stem cell recognition through the immune system of the patient. Thus, preferred is a method according to the present invention, wherein said immune disorder leads to an increase or decrease in the number of immune cells that have reacted to said stem cells in said putative acceptor sample. In one (extreme) example, said immune disorder is SCID.

Yet another aspect of the present invention then relates to a method of diagnosing whether two individual are identical twins, comprising performing a method according to the invention as above, and diagnosing, at least in part, from the number of immune cells that have reacted to said stem cells in said putative acceptor sample, whether donor and acceptor are identical twins. In this context, the lack of a response is indicative for identical twins.

Yet another aspect of the present invention then relates to a method for producing an improved stem cell preparation, comprising a method according to the present invention as above, and selecting a non-reactive or low-reactive sample based, at least in part, on the number of immune cells that have reacted to said stem cells in said putative acceptor sample, and formulating said selected sample into a stem cell preparation.

Another aspect of the present invention then relates to an improved stem cell preparation, produced according to a method according to the invention. In the present invention, the pharmaceutical composition includes pharmaceutical compositions containing a suitable diluent, preservative, solubilizer, emulsifier, adjuvant and/or carrier, together with therapeutically effective amount of the stem cells. In principle, a diluent, buffer, preservative, solubilizer, emulsifier, adjuvant and/or carrier is suitable, if it does not or at least not substantially, interfere with the properties (e.g. quality as above) of the stem cells. The term “therapeutically effective amount” as used in the present specification refers to an amount which provides therapeutic effect on the stipulated conditions and dosage regimen. Such a composition is in a liquid, freeze-dried or dried form, which includes various buffering agents (for example, tris-hydrochloric acid, acetate, phosphate), diluents having various pH and ionicity, additives such as albumin or gelatin to prevent adhesion to the surface, surfactants (for example, Tween 20, Tween 80, Pluronic F68, bile salt), solubilizers (for example, glycerin, polyethylene glycol), antioxidants (for example, ascorbic acid, sodium meta bisulfite), preservatives (for example, thimerosal, benzyl alcohol, parabenes), fillers or isotonic agent (for example, lactose, mannitol), intake of said materials into covalent bondage formation of a polymer such as polyethylene glycol to protein, complex formation with a metal ion, a granular preparation of a polymer compound such as polylactic acid, polyglycolic acid, hydrogel and the like or onto the surface thereof, a nucleus of liposome, microemulsion, micelle minelayer or multilayer follicle, erythrocyte ghost or spheroplast, all as applicable. The examples of specific formulations of the present composition include systemic administering agent for example parenteral, transpulmonary, transnasal and oral, and local administering agents.

The stem cell preparation, produced according to a method according to the invention as employed can further be administered in combination with at least one factor selected from the group consisting of SCF, EGF, EPO, FGF, GM-CSF, G-CSF, IGF-I, IGF-II, TPO, insulin, interferon, interleukin, such as IL-3, IL-6, KGF, M-CSF, PD-ECGF, PDGF, TGF-.alpha. and TGF-beta, and combinations thereof. Other factors can be used with the invention. The factor can be included into the preparation, or is administered concomitantly with said stem cell preparation.

Another aspect of the present invention then relates to method for treating a disease, comprising administering to a patient in need thereof an effective amount of a stem cell preparation as described above. The stem cell preparation can be used to treat a disease such as, for example and without limitation, cancer, diabetes, neurological diseases, stroke, sclerosis, dental diseases, bone damage, heart-related diseases, leukaemia, Parkinson's disease, spinal cord injuries, and muscle damage.

Another aspect of the present invention relates to a method for optimizing stem cell culture media by comparing cell growth in different media using the method according to the present invention. As an example, stem cell preparations that have been raised in media to be tested which show a higher reactivity with immune cells indicate more suitable growth conditions than those with a lower reactivity. The media can be optimized by varying the composition of the media, e.g. by adding or removing slats, buffers or factors.

Finally, still another aspect of the present invention relates to an apparatus that is equipped for performing the diagnostic method(s) according to the present invention as described above. The apparatus can also be equipped with a software that comprises means for performing, at least in part, the steps of the diagnostic method(s) according to the present invention as described above, in particular the generation and display of a graph showing said number of leukocytes counted in each of said plurality of said size-distribution ranges. Preferred is an apparatus that comprises a ROBOCat II device, as available from Cell Science Systems, Ltd., Corp. of Deerfield, Fla., USA.

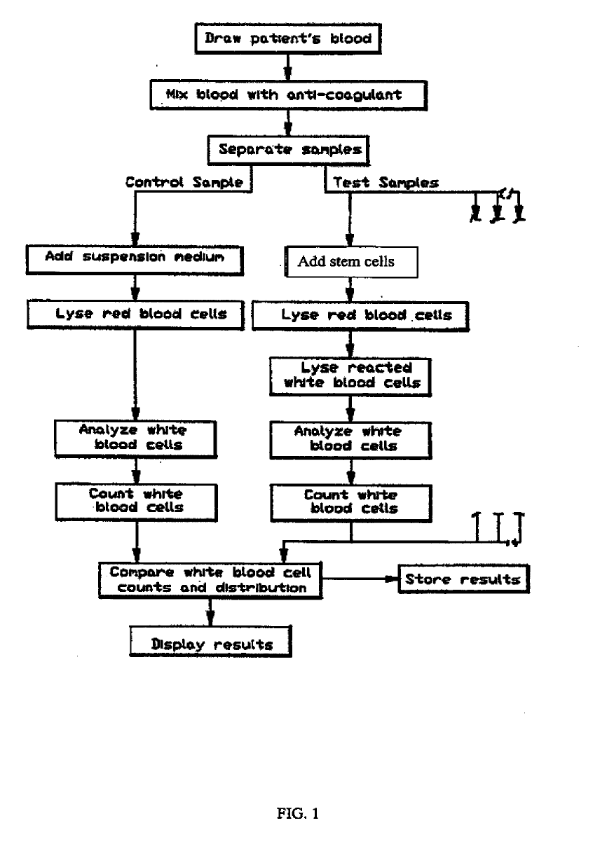

To commence the method of the present invention (generally described in the flow chart of FIG. 1) a blood sample is first drawn from the person being tested (patent, subject, donor). Preferably, 10 cc of oxylated or venous blood is drawn from the subject, using sodium citrate as an anti-coagulant to prevent clotting of the blood during the test. This single sample may be used to perform a wide variety of tests.

It is necessary to secure an even cellular distribution of leukocytes over the entire volume of the blood sample, and so it is necessary to transfer blood to an apparatus (not shown) which will have the capability of continuous flow circulation, in known fashion. Such a device will cause the cells of the sample to be distributed generally evenly over the entire sample.

It is preferred that a single blood sample drawn from the subject be used during a battery of tests for a number of (foreign) stem cell preparations, and so the blood drawn from the subject is separated into a plurality of smaller samples for testing. The preferred method of separation is to place 100 microliters of the drawn blood into a receptacle containing an appropriate suspension medium, e.g. either 1 or 2 ml of a balanced pH saline solution. The precise amount of the suspension medium used is not critical, except that the same amount thereof should be used throughout the test, i.e. in each sample of the same blood. This will ensure that measurements taken for the test and control samples may be directly compared by virtue of the fact that they both have a similar number of leukocytes and volume.

The ten five cc's should be sufficient cubic centimetres of drawn blood prepared as described will be suitable for approximately 150 tests. After separation of the drawn sample into the control and test samples, the stem cells to be tested are introduced into the test samples in the form of solutions as described above for pharmaceutical preparations. Suitable solutions may be purchased commercially from any one of a number of suppliers. In many instances, injectable pyrogen-free, sterilized water or buffer will be suitable. The particular diluting solution selected will depend upon the concentration of the stem cells being mixed, and the selection of an appropriate diluting solution is well within the knowledge of those of ordinary skill in the art. Once made, the mixture is allowed to stand for a suitable period of time (for example 1, 2, 3, 6, up to 24 or even 4 or 5 days) to see the rate of lymphocyte trans-formation (NK cells and CTL's) per hour at a suitable (e.g. room) temperature, or even 37 degrees and then, optionally, filtered through a mesh capable of filtering solid particles, as filtering is not always required.

Different solutions/samples of stem cells can be introduced into the various test samples. It is also preferred that an amount of the suspension medium, equal to that introduced to the test samples, be added to the control sample. This allows the direct comparison of the two samples, since they will have generally identical counts of leukocytes (before the studied reaction), and generally similar volumes.

At this point, each sample, i.e. the control sample and each test sample, will contain a mixture of suspension medium and whole blood, which in turn includes leukocytes, red blood cells and platelets. In addition, the test samples will have the solutions of stem cells therein. The only cells which are of importance to the studied reaction, however, are the leukocytes, and so it is important that counter 12 only count those cells. In the preferred embodiment, counter 12 is ROBOCat II manufactured by Cell Science Systems, Ltd., Corp of Deerfield Beach Fla., which may be set to count the number of particles within a given size range, and is designed to analyze multiple samples sequentially, in automated fashion, as described above. Thus, since platelets are much smaller than leukocytes, it is possible to avoid counting them by setting the minimum particle size at a size greater than that of platelets, and less than that of leukocytes, for example 4.8 microns.

To avoid counting red blood cells, which are of roughly comparable size to the leukocytes, it is preferred that those cells be eliminated. This is preferably accomplished by adding a substance which will immediately cause red blood cells to disintegrate (a “lysing” substance), for example a solution comprising one percent Saponin (Coulter) and the remainder bacteriostatic water. Alternatively, the red blood cells may be mechanically removed from the sample. After the red blood cells and reacted leukocytes are eliminated, preferably after a period of about 30 seconds, and counter 12 is set to the predetermined minimum size level, counter 12 may be used to count and size the leukocytes of the control sample. The results of the counting and sizing of the control sample are used as a reference for comparison of the results of the same counts performed on the test samples.

If the leukocytes in one or more of the test samples recognize any stem cell as harmful, then they will react in the manner described, i.e. by getting larger, then breaking up and finally dissolving. Alternatively, one might expect to see an increase in the number of Natural Killer (NK) cells and cytotoxic lymphocytes (CTL's) that may be active in the rejection process. These reactions will cause a distortion of the size distributions of the leukocytes in the positive test samples, since the number of leukocytes in lower volumetric size ranges will diminish, and the number of leukocytes in higher volumetric size ranges will increase, thereby producing a shift in the size-distributions of the counted leukocytes.

Other methods for determining reactivity between leukocytes and stem cells are possible. It is possible to measure any decrease in the number of donor cells. A decrease could then be related to adverse reactivity with leukocytes. Also, swelling and rupturing of leukocytes could be monitored for indications that the leukocytes are reacting with donor cells. Also, in order to more clearly observe changes in the size or number of host leukocytes at various intervals following co-culture with prospective donor mixtures, it could be beneficial to selectively eliminate the donor cells from the test mixture. In one aspect, this could be accomplished by attaching a cytotoxic agent to one or more monoclonal antibodies that will only recognize surface markers that are expressed on the donor cells. Examples of such cell surface markers include CD 133 receptors expressed on pancreatic islet stem cells, CD 106 and CD 146 expressed on adipote derived mesenchymal stem cells, and CXCR and FLK 1 receptors on embryonic stem cells. Other agents and methods to preferentially lyse or otherwise remove host or donor cells are possible.

The output of counter 12 may be read visually, to determine if the number of white blood cells in the test sample is less than that of the control (indicating positive reaction) or it may be input to analyzer 14, such as the Robocat II analyzer, to obtain cellular population distributions of the number of white blood cells present in each of a plurality of size-distribution ranges. This is referred to as “sizing”.

The output of analyzer 14 may also be input to computer 18 through interface 16, in known fashion, to store the data and automatically compare the results of the count of each test sample to that of the control sample as well as the size-distribution of the white blood cells. If the number of white blood cells in a test sample is less than that of the control by more than the error factor of counter 12, then there is a positive reaction. One might also see an increase in certain lymphocyte sub-fractions, e.g. CTL's, and NK cells. The error factor of each piece of equipment used is readily available from its manufacturer, and may vary from manufacturer to manufacturer. In addition, if the comparison of the size-distribution results indicate enlargement of white blood cells, then there is also a positive reaction. An increase in CTL's and/or NK cells over baseline can also be regarded as a positive reaction. Once all comparisons are made, the output of computer 18 may be displayed by any means desired, such as printer 22 or video display 24, and may also be stored on disc 20 for future reference.

The existence of an appropriate control against which reactivity is measured can be accomplished by a number of different methods. A suspension of donor cells can be added to a suspension of host cells. The combined mixture can be be separated into a number of different samples. The size, number and possibly other characteristics of cells in the entire mixture can be measured before any reaction can occur (T-0). This would be the control. Measurements would then be performed on a sample, or separate samples that are prepared at the same time or from the same initial mixture, at points of time after donor and host cells have been placed together. Any time intervals are possible, but for example T-1 hrs, T-2 hrs, T-6 hrs, T-24 hrs, T-48 hrs, T-72 hrs, and so on. The progress of any reaction between host and donor cells can be viewed as a function of time. The progress of the reaction can be viewed in some cases by examining the donor cells, in other cases by examining the host cells, and in some cases by examining both host and donor cells.

Storing data is useful for later reference on the same subject at a later date. If a control sample of a subject's blood taken at a subsequent time is vastly different from that of a first test, then there may be an indication of a vast change in the state of that subject's immune system. This would suggest further testing to determine the cause of such a change.

It is here noted that the above description was made based on the assumption that stem cells are added to the test samples to produce thereby a reaction.

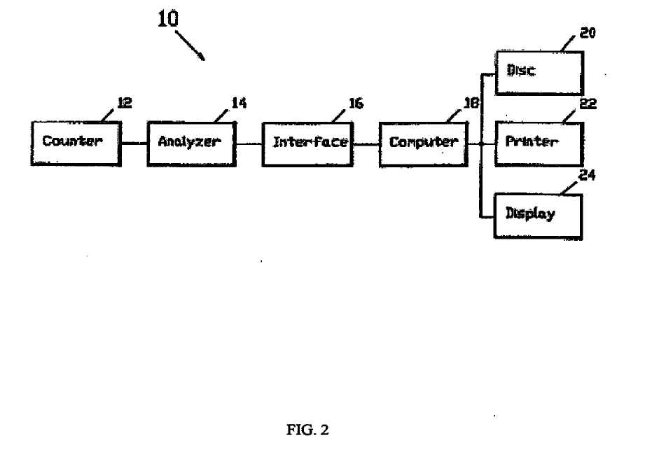

An apparatus in accordance with the present invention, as shown generally at 10 in FIG. 2, comprises a counter 12, an analyzer 14, a computer interface 16 and a computer 18, each connected in series, and a disc 20, a printer 22 and a video display 24, each connected to the output of computer 18.

The invention also has utility in determining the changes that occur to stem cells as the result of various environmental factors that may affect the stem cells positively or adversely. Such factors can be the effects of storage, transportation, preservation, and other factors prior to transplantation. Also, the invention could be used to measure changes in stem cell size and/or number, or proliferative ability, resulting from the presence of various chemical or cellular additives for purposes of therapies, such as drugs like HGH, IGF-1, GM-CSF, treatments, and the like. For example, stimulant A and stimulant B could be chemical signalling compounds that are proposed to stimulate the differentiation of nueronal stem cells into nuerons. The extent of differentiation caused by each of the proposed signalling compounds can then be determined.

The invention also may be used to measure the effectiveness of immune cells from a donor for purposes of treatment of tumors in a host, in a procedure such as allogeneic lymphocyte transplantation. See, for example, Slavin, S. Cancer Immunotherapy with Alloreactive Lymphocytes, New Eng. J. of Med, Vol 343: 802-803 (Sep. 14, 2000). The immune cells, such as Natural Killer (NK) cells or cytotoxic lymphocytes (CTL) from a donor can be added to tumor samples of a host. These can be blood born tumors or in some cases solid tumors that have been biopsied and placed into suspension. The tumor cells can be separated into different samples and then tested against a number of different donor cells to determine which donor cells are most effective at killing or slowing the growth of the tumor cells. This technique could also have utility for treatment of maladies other than tumors. This technique could also be utilized to determine if the proposed treatments cause an adverse immune response in the patient.

Devices and methods as described herein can be used to analyze a composition's ability to destroy cancer cells in a mammalian subject. Cancer cells include those from solid tumors and hematological malignancies. A non-exhaustive list of examples of solid tumor cancers include sarcomas (e.g., fibrosarcoma, rhabdomyosarcoma, osteosarcoma, liposarcoma, neurofibrosarcomas), carcinomas (e.g., those of the bladder, brain, breast, colon, kidney, liver, lung, ovary, pancreas, prostate, stomach, cervix, colon, endometrium, thyroid, ovaries, skin, etc.), melanomas, seminomas, tetratocarcinomas, neuroblastomas and gliomas. A non-exhaustive list of hematological malignancies include multiple myeloma, leukemias (e.g., acute myeloid leukemia, acute lymphocytic leukemia, chronic lymphocytic leukemia, chronic myelogenous leukemia, hairy cell leukemia, etc.), and lymphomas (e.g., chronic lymphocytic lymphoma, lymphoplasmacytic lymphoma, Hodgkin's lymphoma, plasma cell neoplasms, MALT lymphoma, follicular lymphoma, B cell lymphoma, T cell lymphomas, Burketts lymphoma, etc.).

The invention can also be used to measure the immune response to recall antigens, and thereby to measure the effectiveness of different vaccines. Memory cells have surface receptors for certain antigens, for example, tetanus. These memory cells in the presence of the antigen cause the multiplication of immune cells to create an immune response to the antigen. The invention could be used to determine the effectiveness of various vaccines by testing the ability of the host cells to respond, in vitro, to an antigen to which it has been previously exposed, either naturally or through vaccination. The immune response is then determined by measuring the number, size, and/or possibly other characteristics of leukocytes after the introduction of the antigen. By following this procedure with different vaccines, the relative performance of the vaccines can be determined. Further, the speed at which the immune response progresses can be measured to further investigate the relative performance of different vaccines. The procedure could also be used to determine when a patient who at one time may have had a sufficient immune response, no longer has an appropriate immune response and is in need of another vaccine or a booster.

It is also here noted that there is nothing in this description which will limit the application of this method to the human immune system. It is believed that the invention is equally applicable, for example, in the field of veterinary medicine for any animal having an immune system.

As will be readily apparent to those skilled in the art, the above description represents the preferred, but nonetheless illustrative, embodiment of the invention, which may be realized in other specific forms without departing from its spirit or essential characteristics. For example, the entire apparatus may be automated so that once the sample of blood is drawn from the subject there need be no further human intervention or action until the results are complete. Therefore, the full scope of such invention is to be measured by the appended claims, giving thereto the full range of equivalence which comes within the meaning and range of the claims. All patents and publications cited herein are hereby incorporated by reference herein as if each were individually incorporated and fully set forth.

1. A method for diagnosing the compatibility of a biological sample containing stein cells from a donor with the immune system of a recipient, comprising the steps of: a) providing a biological sample containing stem cells from a putative donor, b) providing a biological sample containing immune cells from a putative acceptor of said stem cells, c) combining said samples from a) and b) under conditions that are suitable to allow for a reaction of said stem cells and said immune cells, d) determining the number of immune cells that have reacted to said stem cells in said putative acceptor sample, and e) determining the compatibility of said stem cells of said donor for the acceptor from step d).

2. The method according to claim 1, furthermore comprising determining the number of immune cells that have reacted in said putative acceptor sample before step c).

3. The method according to claim 1, wherein determining the number of immune cells that have reacted in said putative acceptor sample in step d) comprises counting a number of unreacted and reacted leukocytes.

4. The method according to claim 1, wherein determining the number of immune cells that have reacted in said putative acceptor sample in step d) comprises determining the plurality of varying size-distribution ranges of said cells.

5. The method according to claim 1, wherein the immune cells are selected from white blood cells.

6. The method according to claim 1, wherein the stem cells are selected from ES cells, adult stern cells, and/or cells derived from cord blood or amniotic fluid.

7. The method according to claim 1, wherein the stem cells are of the same or a different HLA-type.

8. The method according to claim 1, wherein the stem cells are selected from allogeneic, syngeneic or autologous stem cells.

9. The method according to claim 1, wherein the stem cells are selected from at least one of totipotent, pluripotent, multipotent, oligopotent, quadripotent, tripotent, bipotent, and unipotent stem cells.

10. The method according to claim 1, wherein the stem cells are selected from at least one of cultured ES cells, hematopoietic cells, myeloid precursor cells, mesenchymal progenitor cells, glial-restricted precursor cells, hipotential precursor cells from fetal liver, umbilical cord blood cells, peripheral blood stern cells, cells from amniotic fluid, bone marrow stem cells, and mast cell precursor cells.

11. The method according to claim 1, wherein the biological sample containing stem cells comprises ex vivo multiplicated stem cells.

12. The method according claim 11, wherein the biological sample comprises a stem cell-derived tissue or organ.

13. The method according to claim 1, wherein both the donor and acceptor are a human.

14. The method according to claim 1, further comprising the determination of a stem-cell specific protein expression pattern, in particular of the transcription factors OCT4, SOX2, and/or NANOG.

15. A method for determining the quality of a stem cell preparation, comprising performing a method according to claim 1, and determining the quality of a stem cell preparation, at least in part, from the number of immune cells that have reacted to said stem cells in said putative acceptor sample.

16. The method according to claim 15, wherein said quality is selected from the purity and/or viability of said stem cells.

17. A method of diagnosing an immune disorder affecting stem cell recognition comprising performing a method according to claim 1, and diagnosing said immune disorder, at least in part, from the number of immune cells that have reacted to said stem cells in said putative acceptor sample.

18. The method according to claim 17, wherein said immune disorder leads to an increase or decrease in the number of immune cells that have reacted to said stem cells in said putative acceptor sample.

19. The method according to claim 17, wherein said immune disorder is SCID.

20. A method for producing an improved stem cell preparation, comprising a method according to claim 1, and selecting a non-reactive or low-reactive sample based, at least in part, on the number of immune cells that have reacted to said stem cells in said putative acceptor sample, and formulating said selected sample into a stem cell preparation.

21. A stem cell preparation, produced according to claim 20.

22. A method for treating a disease, comprising administering to a patient in need thereof an effective amount of a stem cell preparation according to claim 21.

23. The method according to claim 22, wherein said disease is selected from Cancer, diabetes, neurological diseases, stroke, sclerosis, dental diseases, bone damage, heart-related diseases, leukemia, Parkinson's disease, spinal cord injuries, and muscle damage.

24. An apparatus, equipped for performing the method according to claim 1.

25. The apparatus according to claim 24, which comprises a ROBOCat II element.

26. A method for measuring cell reactivity between allogeneic donor and host cells, comprising the steps of mixing the allogeneic cells, incubating the mixture, and adding to the mixture a cytotoxic agent that is preferential to one of either the host or the donor cells, and then measuring the reactivity of the other of either the host or the donor cells.

27. The method of claim 26, wherein said cytotoxic agent is attached to at least one monoclonal antibody that will recognize only surface markers on one of either the host or the donor cells, but not the other of either the host or the donor cells.

28. The method of claim 27, wherein said cell surface markers comprise at least one selected from the group consisting of CD 133 receptors expressed on pancreatic islet stem cells, CD 106 and CD 146 expressed on adipote derived mesenchymal stem cells, and CXCR and FLK 1 receptors on embryonic stem cells.

29. A method for measuring the reactivity between allogeneic host and donor cells, comprising the steps of mixing the host and donor cells and measuring the number of cells, allowing the mixture to incubate for a period of time, and then measuring the cells after said period of time.

30. The method of claim 29, wherein said measuring step comprises counting the number of cells.

31. The method of claim 29, wherein said measuring step comprises measuring the size of the cells.

32. The method of claim 29, further comprising the step of allowing said mixture to incubate for additional periods of time, and then measuring said cells after said additional periods of time.

33. A method for determining changes to stem cells as the result of environmental factors, comprising the steps of exposing said cells to said environmental factors, and measuring the effect of said environmental factors on said cells after said exposure.

34. The method of claim 33, wherein said environmental factors comprise at least one selected from the group consisting of cell storage, transportation, and preservation.

35. The method of claim 33, wherein said environmental factors comprise the presence of at least one selected from the group consisting of chemical or cellular additives for purposes of therapies.

36. The method of claim 35, wherein said factor is selected from the group consisting of human growth hormone, IGF-I, and GM-CSF.

37. The method of claim 33, wherein said step of measuring comprises measuring at least one of cell size or number.

38. The method of claim 33, wherein said step of measuring comprises determining the extent of differentiation of said cells.

39. The method of claim 33, further comprising the step of exposing different allotments of said cells to at least two environmental factors, measuring the effect of each of said environmental factors on said cells, and then comparing the effectiveness of said environmental factors on said cells.

40. A method of treating disease in a host patient, comprising the steps of providing donor cells selected to have a therapeutic effect on said disease, obtaining diseased cells from said patient, mixing the diseased cells with said therapeutic donor cells, and measuring the effect of said therapeutic donor cells on said diseased cells.

41. The method of claim 40, wherein said diseased cells are target cells.

42. The method of claim 41, wherein said diseased cells are tumor cells.

43. The method of claim 40, wherein said therapeutic donor cells are immune cells.

44. The method of claim 43, wherein said immune cells are selected from Natural Killer cells and cytotoxic cells.

45. The method of claim 40, wherein said providing step comprises proving at least two different types of donor cells, mixing different allotments of said host cells with said different types of therapeutic donor cells, measuring the effect of said therapeutic cells on said host cells, and comparing said effect to determine a relative effectiveness of said different types of donor cells on said host cells.

46. The method of claim 45, wherein said host cells are disease target cells.

47. The method of claim 40, further comprising the step of administering to a patient a preparation containing therapeutic donor cells determined to be most effective against said host cells.

48. A therapeutic preparation prepared by the method of claim 40.

49. A method for measuring the immune response of a patient to recall antigens, comprising the steps of obtaining a blood sample from said patient, exposing said sample to said recall antigen, and measuring the effect of said exposure on immune cells of said patient in said sample.

50. The method of claim 49, wherein said measuring step comprises counting the number of cells.

51. The method of claim 49, wherein said measuring step comprises measuring the size of the cells.
